(19) United States
(12) Patent Application Publication
(10) Pub. No.: US 2014/0109903 A1
(43) Pub. Date: Apr. 24, 2014

(54) OROGASTRIC TUBE GUIDES AND METHODS OF USING THE SAME

(71) Applicant: Tariq Chaudhry, Tampa, FL (US)

(72) Inventor: Tariq Chaudhry, Tampa, FL (US)

(73) Assignee: H. Lee Moffitt Cancer Center and Research Institute, Inc., Tampa, FL (US)

(21) Appl. No.: 14/113,268

(22) PCT Filed: Apr. 20, 2012
(86) PCT No.: PCT/US12/34380

§ 371 Date: Dec. 6, 2013

Related U.S. Application Data
(60) Provisional application No. 61/478,179, filed on Apr. 22, 2011.
(60) Provisional application No. 61/511,292, filed on Jul. 25, 2011.

(51) Int. Cl. A61J 15/00; A61M 16/04

(52) U.S. Cl. CPC A61J 15/0003; A61M 16/0463
USPC 128/202.15; 604/528; 604/516

(57) ABSTRACT

The present application relates to orogastric tube guides and to related systems and methods. The orogastric tube guides and systems can be used to direct an orogastric tube into the esophagus of a patient during any medical or surgical procedure where placement of an orogastric tube is indicated or used.

### CROSS-REFERENCE TO RELATED APPLICATIONS

This application claims the benefit of U.S. Provisional Patent Application No. 61/478,179, filed Apr. 22, 2011 and U.S. Provisional Patent Application No. 61/511,292, filed Jul. 25, 2011, both of which are incorporated herein by reference in their entirety.

### TECHNICAL FIELD

The present application relates to orogastric tube guides and to the guiding of orogastric tubes into the esophagus.

### BACKGROUND

A vast majority of patients undergoing general anesthesia require orogastric tube placement to decompress and drain gastric secretions for better surgical visualization and to minimize the risk of aspiration of gastric contents.

The traditional placement technique is blind, traumatic and unhygienic as gastric secretions are a source of cross-contamination. Orogastric tubes have an inherent rigidity favoring insertion. Unsuccessful insertion attempts lead, however, to softening of the tube by the higher oral temperature compared to the ambient temperature. This leads to curling of the tube in patient's mouth rather than going down the esophagus resulting in product waste in addition to the complications resulting from multiple placement attempts.

### SUMMARY

The present application relates to orogastric tube guides and to related systems and methods. The orogastric tube guides and systems can be used to direct an orogastric tube into the esophagus of a patient during any medical or surgical procedure where placement of an orogastric tube is indicated or used.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1 is a schematic illustration of an example orogastric tube guide and endotracheal tube.

FIG. 2 is a schematic illustration of an example endotracheal tube placed in a typical patient-inserted orientation.

FIG. 3 is a schematic illustration of an example orogastric tube guide and endotracheal tube positioned in a subject.

FIGS. 4A-D are schematic illustrations of an example orogastric tube guide and endotracheal tube and aspects thereof.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

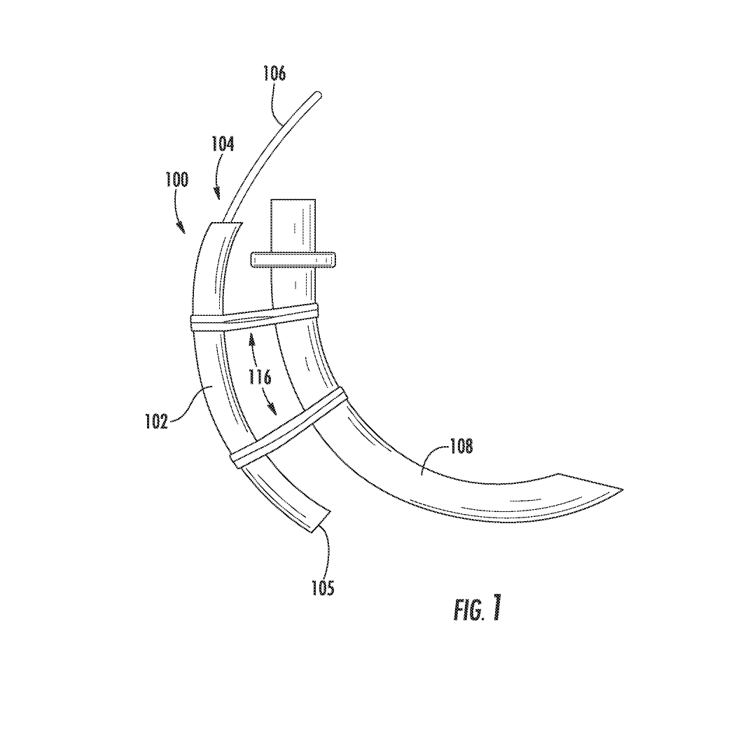

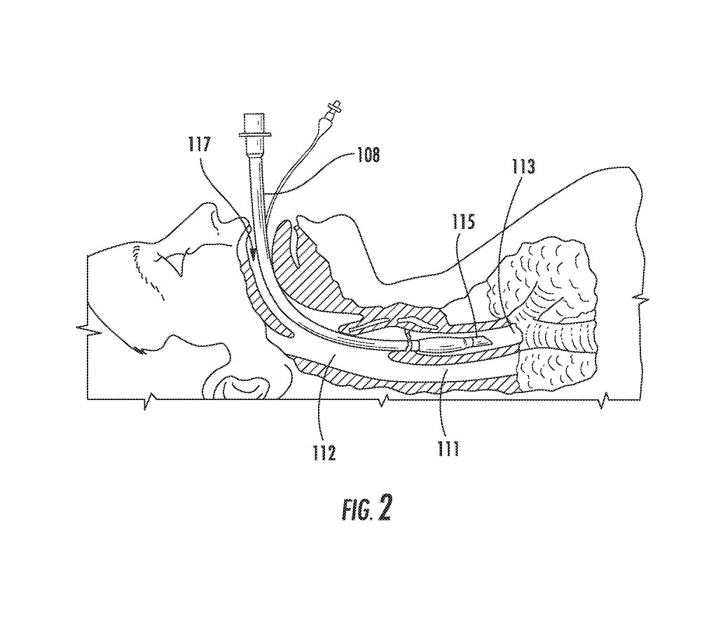

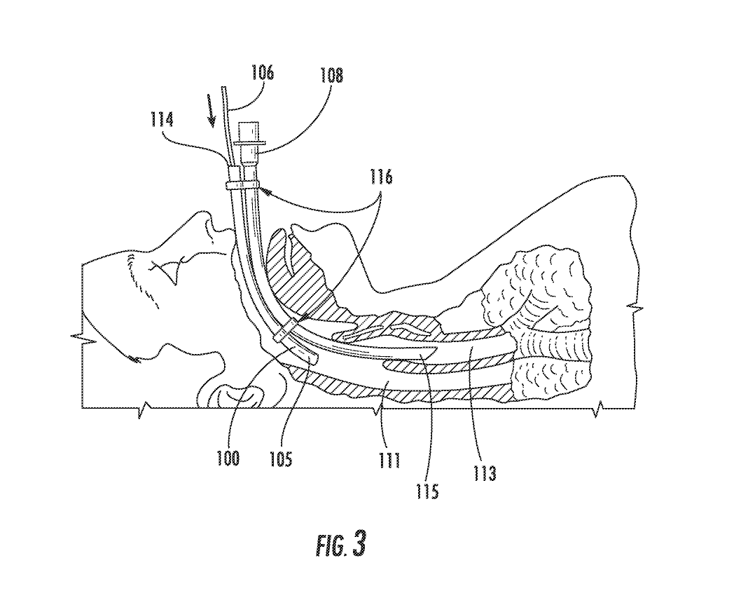

Referring to FIGS. 1-3, an orogastric tube guide 100 is illustrated. The orogastric tube guide 100 can be used to direct an orogastric tube 106 into the esophagus 111 of a subject.

The orogastric tube guide 100 comprises a conduit 102 having a passage 104 sized to allow for the slidable advancement of an orogastric tube 106 there through the passage. For example, the passage 104 can have an opening and inner-luminal diameter along the length of the conduit that allows an operator to slide an orogastric tube through the passage. The size of the passage 104 may vary based on the size of the orogastric tube 106 that is used in a given medical procedure.

For example, the passage can be sized to allow any size orogastric tube 106 to be passed through it. Optionally, the conduit passage is sized to allow the slidable passage of an orogastric tube 106 that is up to 18 french in size.

Optionally, the diameter of the passage 104 is matched closely to the outside diameter of the orogastric tube used in a medical procedure. For example, the luminal diameter of the guide may in some cases be just larger than the outer diameter of the orogastric tube used in the procedure such that slidable passage is achieved while the inner luminal walls of the conduit passage are in close approximation, or even in contact, with the outside of the orogastric tube. The conduit can also vary in length. Optionally, the conduit is 9 inches long or shorter.

The conduit 102 of the orogastric tube guide 100 is secured or securable to an endotracheal tube 108. When secured to the endotracheal tube 108, the conduit 102 is optionally oriented such that the passage 104 is posterior to the endotracheal tube 108 when the endotracheal tube is in its patient-inserted orientation. Patient-inserted orientation refers to an endotracheal tube inserted and/or positioned into the trachea of subject as typical for use in medical and surgical procedures.

The conduit 102 is configured to guide an orogastric tube into the esophagus of a subject when the conduit 102 is secured to the endotracheal tube 108 and when the endotracheal tube or a portion thereof is located in the trachea of the subject. The orogastric tube guide 100 can therefore be used to direct an orogastric tube into the esophagus 111 while an endotracheal tube is positioned in the trachea 113.

When the conduit 102 is placed in a subject, at least a portion of the conduit 102 can be located in the pharyngeal cavity 112 of the subject. For example, the distal end 105 of the conduit 102 can be located in the pharyngeal cavity 112 of the subject. The distal end 105 comprises an opening that allows the passage of the distal end of the orogastric tube 106 out of the conduit and into the esophagus 111. The pharyngeal cavity includes, for example, the oropharynx or laryngopharynx.

At least a portion 114 of the conduit 102 can also be located outside of the pharyngeal cavity and, optionally, outside of the oral cavity of the subject. This portion 114 also has an opening for insertion of the orogastric tube 106 into the conduit passage 104.

The conduit 102 is optionally secured to the endotracheal tube 108 by at least one connector 116. Optionally, the conduit 102 is releaseably secured or securable to the endotracheal tube 108. For example, the at least one connector 116 can be used to attach the conduit 102 to the endotracheal tube 108 and the at least one connector can also release the attachment to allow the separation of the endotracheal tube 108 from the conduit 102. The conduit is optionally flexible. In some examples, the connector is a clamp, such as a snap-on clamp that can snap onto the guide 100 and/or endotracheal tube using a friction fit or other attachment mechanism. The guide 100 and other portions of the devices and systems described herein can optionally comprise material used in medical applications such as medical tubing applications. For example, the guide can be made of the same or similar material to the endotracheal tube. The guide optionally comprises medically compatible plastic or polymeric material. Optionally, the guide is left in the subject over a period of time, for example, for the duration of a medical or surgical procedure. Optionally, the conduit 102 is spaced from the endotracheal tube 108 when the conduit is secured to the endotracheal tube.

Still referring to FIGS. 1-3, further provided is a system that comprises an endotracheal tube 108 and an orogastric tube guide 100. The orogastric tube guide 100 comprises a conduit 102 configured for attachment to the endotracheal tube 108 and the conduit 102 has a passage that is sized to allow for slidable advancement of an orogastric tube there through the passage.

The system optionally further comprises an orogastric tube 106 wherein the orogastric tube is configured to be slidably advanced through the passage 104 of the conduit 102. As described above, the conduit 102 can be attached to the endotracheal tube 108 such that its passage is posterior to the endotracheal tube when the endotracheal tube is in its patient-inserted orientation. The conduit 102 is configured, and can be used, to guide an orogastric tube into the esophagus 111 of a subject when the conduit is attached to the endotracheal tube 108 and when the endotracheal tube, or a portion thereof, is located in the trachea of the subject.

Also provided are methods for placing an orogastric tube 106 into the esophagus 111 of a subject. The methods include providing an endotracheal tube 108 and an orogastric tube guide 100. The orogastric tube guide 100 comprises a conduit 102 having a passage that is sized to allow the slidable passage of an orogastric tube 106 there through the passage.

An end 115 of the endotracheal tube and an end 105 of the conduit can be inserted into the oral cavity of the subject and positioned such that the end 115 of the endotracheal tube is located in the trachea of the subject and the end 105 of the conduit is located in the pharyngeal cavity of the subject. The pharyngeal cavity includes, for example, the oropharynx or laryngopharynx.

The orogastric tube 106 is inserted into the passage of the conduit and advanced through the passage into the esophagus 111 of the subject. The conduit 102 can be secured to the endotracheal tube 108 prior to inserting the end of the endotracheal tube and conduit into the oral cavity. Optionally, the conduit is secured to the endotracheal tube in a position posterior to the endotracheal tube. Optionally, a second end 114 of the conduit is positioned outside of the oral cavity. The end 114 of the conduit positioned outside of the oral cavity has an opening where the orogastric tube is inserted into the conduit.

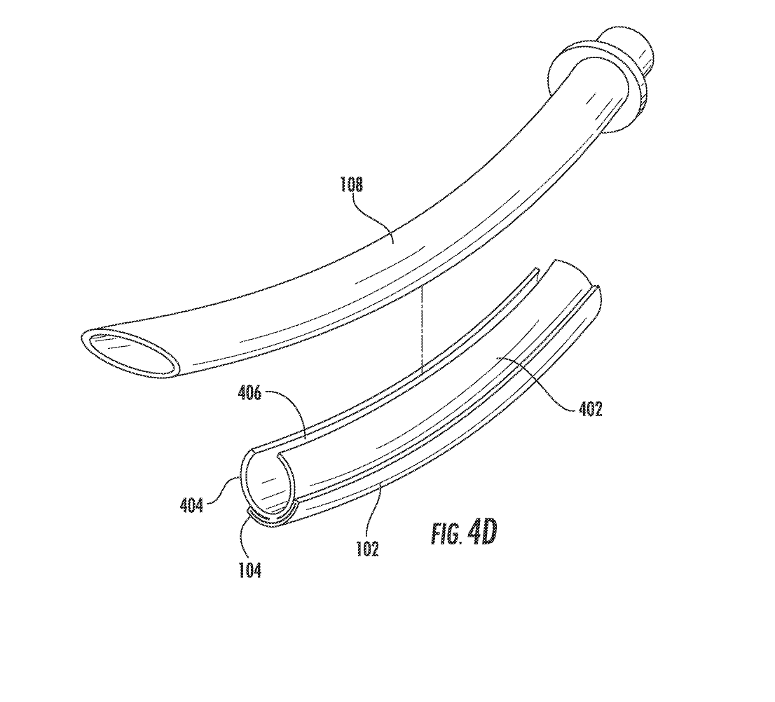

Referring now to FIG. 4, an orogastric tube guide 400 comprises a conduit 102 having a passage 104. At least a portion of the passage 104 is moveable between a first collapsed state shown in FIG. 4B and a second expanded state shown in FIG. 4C. The second expanded state is configured to allow for slidable advancement of an orogastric tube there through the passage 104. Thus, in the expanded state, the lumen of the passage is sufficiently open to allow slidable passage of the orogastric tube.

The conduit 102 is secured or securable to an endotracheal tube 108. Optionally, the conduit 102 comprises a pliable or flexible material that tends to a collapsed state when not held or actuated into the expanded state. Such materials may include, for example, rubber or another elastic material, such as an elastic polymer.

The passage 104 optionally maintains the collapsed state prior to slideable advancement of the orogastric tube. Thus, the passage can be expanded to the expanded state on insertion and advancement of an orogastric tube and the passage can return to the collapsed state on removal of the orogastric tube. Optionally, the collapsed state prevents or impedes substantial fluid flow there through the passage.

Also provided are systems, comprising an endotracheal tube and an orogastric tube guide 400, wherein the orogastric tube guide 400 comprises a conduit 102 having a passage 104. At least a portion of the passage 104 is optionally moveable between a first collapsed state (FIG. 4B) and a second expanded state (FIG. 4C). The second expanded state is configured to allow for slidable advancement of an orogastric tube there through the passage.

The orogastric tube guide 400 comprises a conduit 102 having a passage 104 sized, in its expanded state, to allow for the slidable advancement of an orogastric tube 106 there through the passage. For example, the expanded passage 104 can have an opening and inner-luminal diameter along the length of the conduit that allows an operator to slide an orogastric tube through the passage. The size of the expanded passage 104 may vary based on the size of the orogastric tube 106 that is used in a given medical procedure.

For example, the expanded passage can be sized to allow any size orogastric tube 106 to be passed through it. Optionally, the conduit expanded passage is sized to allow the slidable passage of an orogastric tube 106 that is up to 18 french in size.

Optionally, the diameter of the expanded passage 104 is matched closely to the outside diameter of the orogastric tube used in a medical procedure. For example, the luminal diameter of the guide may in some cases be just larger than the outer diameter of the orogastric tube used in the procedure such that slidable passage is achieved while the inner luminal walls of the conduit passage are in close approximation, or even in contact, with the outside of the orogastric tube. The conduit can also vary in length. Optionally, the conduit is 9 inches long or shorter.

As described with reference to FIGS. 1-3, the conduit 102 of the orogastric tube guide 400 is secured or securable to an endotracheal tube 108. When secured to the endotracheal tube 108, the conduit 102 is optionally oriented such that the passage 104 is posterior to the endotracheal tube 108 when the endotracheal tube is in its patient-inserted orientation. Patient-inserted orientation refers to an endotracheal tube inserted and/or positioned into the trachea of subject as typical for use in medical and surgical procedures.

The conduit 102 is configured to guide an orogastric tube into the esophagus of a subject when the conduit 102 is secured to the endotracheal tube 108 and when the endotracheal tube or a portion thereof is located in the trachea of the subject. The orogastric tube guide 400 can therefore be used to direct an orogastric tube into the esophagus 111 while an endotracheal tube is positioned in the trachea 113.

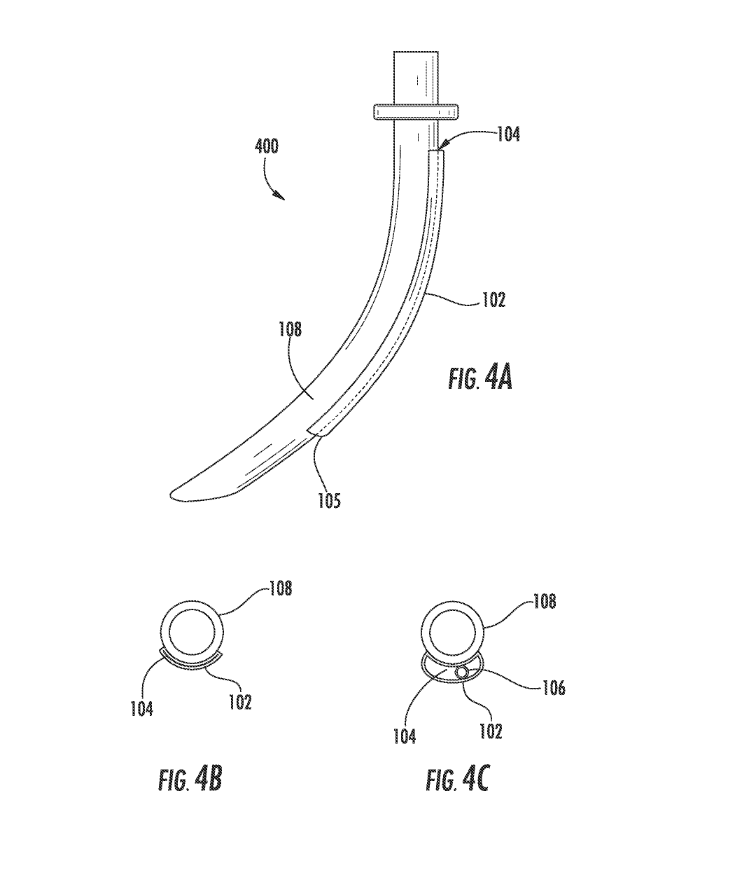

As described with reference to FIG. 3, when the conduit 102 is placed in a subject, at least a portion of the conduit 102 can be located in the pharyngeal cavity 112 of the subject. For example, the distal end 105 of the conduit 102 can be located in the pharyngeal cavity 112 of the subject. The distal end 105 comprises an opening that allows the passage of the distal end of the orogastric tube 106 out of the conduit and into the esophagus 111. The pharyngeal cavity includes, for example, the oropharynx or laryngopharynx.

At least a portion 114 of the conduit 102 can also be located outside of the pharyngeal cavity and, optionally, outside of the oral cavity of the subject. This portion 114 also has an opening for insertion of the orogastric tube 106 into the conduit passage 104.

Referring to FIG. 4D, the conduit 102 is optionally secured to the endotracheal tube 108 by at least one connector 406. Optionally, the conduit 102 is releaseably secured or securable to the endotracheal tube 108 using, for example the connector 402. For example, the at least one connector 406 can be used to attach the conduit 102 to the endotracheal tube 108 and the at least one connector can also release the attachment to allow the separation of the endotracheal tube 108 from the conduit 102.

The connector can extend along the length, or a portion of the length, of the conduit 102. For example, the connector 406 can extend along the length of the conduit 102 and can include a first side 404 and second side 402, which are separated by a slot. The endotracheal tube may be forcibly pushed into the connector through the slot such that the slot expands to receive the endotracheal tube. Because the slot is narrower than the diameter within the connector 406, and because the slot is resilient, it can snap back to its original width once the endotracheal tube is positioned in the connector 406. Moreover, the connector 406 optionally has an inner diameter that is less than the outer diameter of the endotracheal tube 108. In this case, the force of the connector trying to regain its original dimensions following the insertion of the endotracheal tube optionally functions to secure the endotracheal tube within the connector 406.

The conduit is optionally flexible. In some examples, as with the connector 406, the connector is a clamp, such as a snap-on clamp that can snap onto the conduit 102 and/or endotracheal tube using a friction fit or other attachment mechanism. The conduit 102 and other portions of the devices and systems described herein can optionally comprise material used in medical applications such as medical tubing applications. For example, the guide can be made of the same or similar material to the endotracheal tube. The guide optionally comprises medically compatible plastic or polymeric material. Optionally, the guide is left in the subject over a period of time, for example, for the duration of a medical or surgical procedure. Optionally, the conduit 102 is spaced from the endotracheal tube 108 when the conduit is secured to the endotracheal tube.

The device shown in FIG. 4 and the systems described above can be used to place an orogastric tube into the esophagus of a subject. For example, an endotracheal tube may be provided and the orogastric tube guide may be provided. An end of the endotracheal tube and the conduit are inserted into the oral cavity of the subject such that the end of the endotracheal tube is located in the trachea of the subject and the end of the conduit is located in the pharyngeal cavity of the subject. An orogastric tube is inserted into the passage of the conduit, expanding the passage, and advancing the orogastric tube through the passage and into the esophagus of the subject.

The orogastric tube guides, systems, and methods described herein can optionally take advantage of an endotracheal tube placed in a subject for general anesthesia. The endotracheal tube acts as a guide for orogastric tube placement. As described throughout, the orogastric tube guides comprise a hollow conduit with the internal diameter wide enough to channel the orogastric tube through it. This orogastric tube guides may be attached to the backside of the endotracheal tube, prior to intubation. After intubation, the orogastric tube is inserted through an open end of the orogastric tube guide and since the esophagus is behind and follows the same curvature as the trachea, the tube slides through the esophagus into the stomach.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An orogastric tube guide, comprising:
a conduit having a passage, wherein at least a portion of the passage is moveable between a first collapsed state and a second expanded state, wherein the second expanded state is configured to allow for slidable advancement of an orogastric tube there through the passage, and wherein the conduit is secured or securable to an endotracheal tube.

2. The orogastric tube guide of claim 1, wherein the passage maintains the collapsed state prior to slideable advancement of the orogastric tube.

3. The orogastric tube guide of claim 1, wherein the passage expands to the expanded state on insertion and advancement of the orogastric tube.

4. The orogastric tube guide of claim 3 wherein the passage returns to the collapsed state on removal of the orogastric tube.

5. The orogastric tube guide of claim 1, wherein the collapsed state prevents or impedes substantial fluid flow there through the passage.

6. The orogastric tube guide of claim 1, wherein the conduit is secured or securable such that the passage is posterior to the endotracheal tube when the endotracheal tube is in its patient-inserted orientation.

7. The orogastric tube guide of claim 1, wherein the conduit is configured to guide the orogastric tube into the esophagus of a subject when the conduit is secured to the endotracheal tube and when the endotracheal tube or a portion thereof is located in the trachea of the subject.

8. The orogastric tube guide of claim 1, wherein at least a portion of the conduit is located in the pharyngeal cavity of the subject.

9. The orogastric tube guide of claim 8, wherein at least a portion of the conduit is located outside of the pharyngeal cavity of the subject.

10. The orogastric tube guide of claim 1, wherein the conduit is secured to the endotracheal tube by at least one connector.

11. The orogastric tube guide of claim 1, wherein the conduit is releaseably secured or securable to the endotracheal tube.

12. The orogastric tube guide of claim 1, wherein the conduit passage is sized to allow the slidable passage of an orogastric tube that is up to 18 french in size.

13. The orogastric tube guide of claim 1, wherein the conduit is 9 inches long or shorter.

14. The orogastric tube guide of claim 1, wherein the conduit is flexible.

15. The orogastric tube guide of claim 1, wherein the conduit is spaced from the endotracheal tube when the conduit is secured to the endotracheal tube.

16. A system, comprising:
a. an endotracheal tube; and
b. an orogastric tube guide, wherein the orogastric tube guide comprises a conduit having a passage, wherein at least a portion of the passage is moveable between a first collapsed state and an second expanded state, wherein the second expanded state is configured to allow for slidable advancement of an orogastric tube there through the passage, and wherein the conduit is secured or securable to an endotracheal tube.

17. The system of claim 16, wherein the passage maintains the collapsed state prior to slideable advancement of the orogastric tube.

18. The system of claim 1, wherein the passage expands to the expanded state on insertion and advancement of the orogastric tube.

19. The system of claim 18 wherein the passage returns to the collapsed state on removal of the orogastric tube.

20. The system of claim 16, wherein the collapsed state prevents or impedes substantial fluid flow there through the passage.

21. The system of claim 16, further comprising an orogastric tube wherein the orogastric tube is configured to be slidably advanced through the passage of the conduit.

22. The system of claim 16, wherein the conduit is attached to the endotracheal tube such that its passage is posterior to the endotracheal tube when the endotracheal tube is in its patient-inserted orientation.

23. The system of claim 16, wherein the conduit is configured to guide the orogastric tube into the esophagus of a subject when the conduit is attached to the endotracheal tube and when the endotracheal tube or a portion thereof is located in the trachea of the subject.

24. The system of claim 23, wherein at least a portion of the conduit is located in the pharyngeal cavity of the subject.

25. The system of claim 23, wherein at least a portion of the conduit is located outside of the pharyngeal cavity of the subject.

26. The system of claim 16, further comprising at least one connector for attaching the conduit to the endotracheal tube.

27. A method of placing an orogastric tube into the esophagus of a subject, comprising:
a. providing an endotracheal tube;
b. providing an orogastric tube guide, wherein the orogastric tube guide comprises a conduit having a passage, wherein at least a portion of the passage is moveable between a first collapsed state and an second expanded state, wherein the second expanded state is configured to allow for slidable advancement of an orogastric tube there through the passage, and wherein the conduit is secured or securable to an endotracheal tube;
c. inserting an end of the endotracheal tube and the conduit into the oral cavity of the subject such that the end of the endotracheal tube is located in the trachea of the subject and the end of the conduit is located in the pharyngeal cavity of the subject; and
d. inserting the orogastric tube into the passage of the conduit, expanding the passage, and advancing the orogastric tube through the passage and into the esophagus of the subject.
